(19) United States
(12) Patent Application Publication
(10) Pub. No.: US 2021/0187141 A1
(43) Pub. Date: Jun. 24, 2021

(54) METHOD AND APPARATUS FOR STERILIZING ENDOSCOPES

(71) Applicant: CISA PRODUCTION S.R.L., Lucca (IT)

(72) Inventor: Francesco CROTTI, Lucca (IT)

(21) Appl. No.: 16/755,566

(22) PCT Filed: Oct. 9, 2018
(86) PCT No.: PCT/IT2018/050188

§ 371 Date: Apr. 11, 2020

(30) Foreign Application Priority Data
Oct. 13, 2017 (IT) 102017000115493

(51) Int. Cl. A61L 2/18; B08B 3/10; A61B 90/70; A61B 1/12; A61L 2/26

(52) U.S. Cl. CPC A61L 2/18; B08B 3/10; A61B 90/70; A61B 1/123; A61L 2202/123; A61B 2090/701; A61L 2202/17; A61L 2202/24; A61L 2202/18; A61L 2/26

(57) ABSTRACT

A method for the sterilization of endoscopes and similar instruments, comprising the following steps of: washing and/or disinfecting or sterilizing an endoscope in a container by one or more washing fluids; dry the endoscope inside the container using one or more drying fluids. The method is characterized by the fact that the pressure inside the container during disinfection or sterilization and/or drying is kept lower than atmospheric pressure and the container is pressurized after drying, at a pressure higher than at atmospheric pressure, before releasing the endoscope.

The present invention relates to an apparatus and a method for the sterilization of endoscopes. In particular, the apparatus and the method of the invention allow the execution of washing, disinfection, sterilization and drying of endoscopes in a single operating station, providing the instrument sterilized and dried inside a hermetic container.

It is known that the use of endoscopes requires frequent washing and disinfection operations due to the daily use of the same endoscope on several patients. On some of these instruments the sterilization is required, which involves further steps, among which the packaging and the treatment inside a low temperature sterilizer.

Traditional multi-stage procedures are known which provide for the execution of washing, disinfection and sterilization in different phases and/or operating stations; in practice, with these systems it is not possible to perform a single treatment cycle to wash, disinfect, sterilize and package an endoscope using a single operative process. The disadvantages of these systems and the consequent deficiencies in terms of safety and functionality are self-evident.

A solution to the aforementioned drawbacks was provided by EP1696969B1 which discloses a system for the washing, disinfection, sterilization, drying and storage of endoscopes which provides for the use of a hermetically sealed container, in which a containment compartment for an endoscope is formed. This container is provided with a multiple connector that allows the connection of the endoscope channels with a system of external pumps through which washing and sterilization fluids are fed into the same channels. The multiple connector also allows to feed a gas for emptying or drying said channels. The system, in addition to the container, consists of two machines, the first for washing, disinfection, sterilization, the second for hot air drying.

The present invention relates to an apparatus and a method like those described in the aforementioned patent, with the aim to further increase its effectiveness and ease of use, providing further advantages both from the point of view of safety and for what concerns the realization of the apparatus and the implementation of the method. This result has been achieved, in accordance with the present invention, by adopting the idea of providing an apparatus and a method having the characteristics indicated in the independent claims. Other characteristics of the present invention are the subject of the dependent claims.

An apparatus and a method for washing, disinfecting, sterilizing, drying and storing endoscopes in accordance with the present invention has improved utilization characteristics, i.e. a more effective execution of the washing, disinfection, sterilization, drying and storage steps, with the achievement of a higher degree of safety, in relation to the sterility of the treated instruments, the environments in which the treatment takes place, and the operators involved in the treatment of the instruments.

Among the advantages of the present invention, the following can be listed as non-limiting advantages: the apparatus and the method allow to greatly increase the safety related to the sterility of the container inside which the sterilized instrument is housed thanks to a pressurization of the container which determines the substantial impossibility of undetected contamination; the apparatus and the method allow to significantly increase the efficiency of the drying process thanks to the realization of the drying phase under conditions of pressure lower than the atmospheric pressure; the apparatus and the method make it possible to increase the effectiveness of the sterilization process to a very high degree, thanks to the realization of the drying phase under conditions of pressure lower than the atmospheric pressure; the apparatus and method make it possible to reduce the consumption of sterilizing liquids by implementing the sterilization phase at a pressure lower than the atmospheric pressure; the apparatus and the method allow an additional cleaning of the connection ducts between the apparatus and the container thanks to the advantageous action deriving from the so-called pulsed vacuum; the container usable in an apparatus according to the invention is advantageously provided with an indicator of the internal pressure able to indicate anomalies related to pressure values that are lower with respect to a predetermined alarm value; the apparatus and method allow the use of a container provided with a support for vertical positioning, both inside a sterilizing machine, and during storage; it is possible to use a container provided with at least two openings for its connection to the sterilizer machine so as to optimize the washing and sterilization processes, with an easy drainage; it is possible to use a container provided with connection openings at the top and bottom to allow a bottom-up filling of the same container with the complete immersion of the endoscope, substantially reducing to zero the possibility of non-immersed portions of the endoscope and guaranteeing a constant outflow of air upwards; it is possible to use a container provided with at least one internal diverter to create a movement of the washing fluid capable of increasing the efficiency of the treatment; it is possible to use a container provided with an integrated handle to improve the ease and efficiency of the transport, as well as its handling during insertion and extraction from treatment and/or storage apparatus; it is possible to use a container provided with a radio frequency or RFID type identification element which allows its unambiguous identification; with the present invention, it is possible to control flow and pressure independently; moreover, the present invention can provide a “trough the wall” passage or a passage between two zones with different sterility coefficient.

These and further advantages and characteristics of the present invention will be better understood by any person skilled in the art, thanks to the following description and to the attached drawings, provided as an example but not to be considered in a limitative sense, in which:

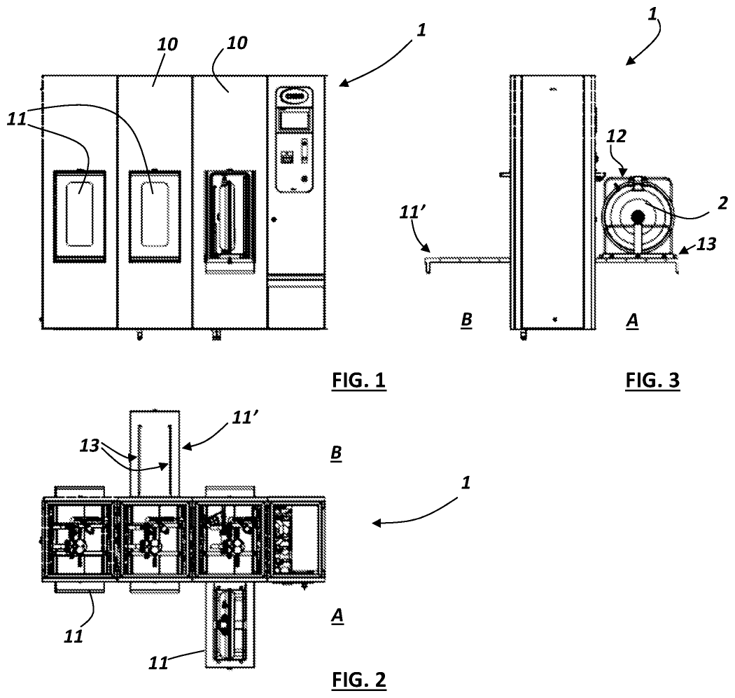

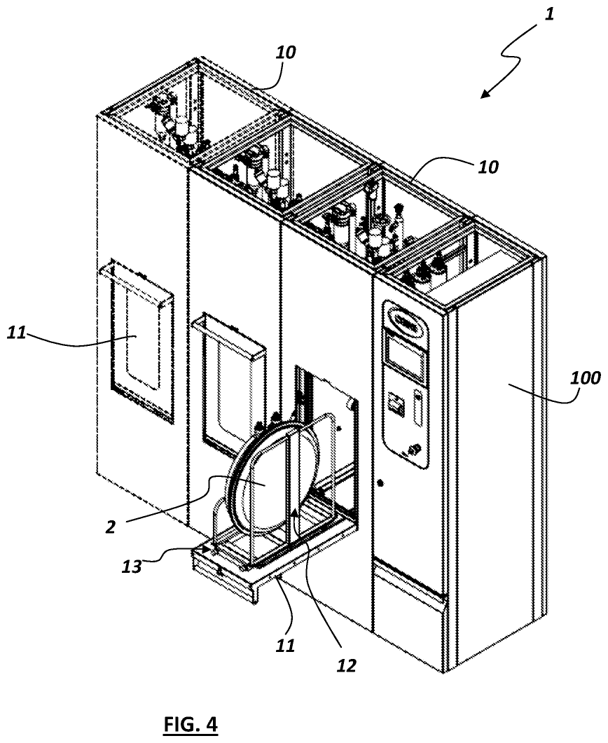

FIGS. 1, 2, 3 and 4 relate to a possible embodiment of an apparatus for washing, sterilizing and drying endoscopes, represented respectively in a front view (FIG. 1), in a plan view from above (FIG. 2), in a side view (FIG. 3) and in a perspective view (FIG. 4);

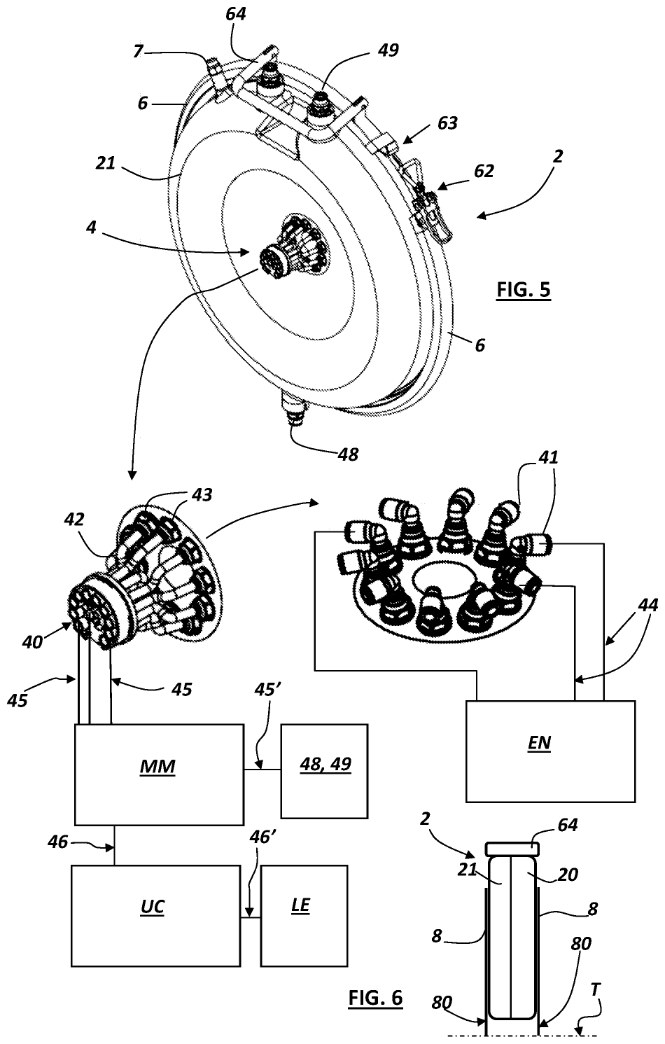

FIG. 5 relates to a possible embodiment of a container for washing, disinfection, sterilization, drying and storage of endoscopes, represented respectively in an overall perspective view with an enlarged detail, where the same detail is represented according to two different views; and

FIG. 6 is a schematic side view of another possible embodiment of a container, provided with a support structure usable in combination with the apparatus in question.

The apparatus and the method of the present invention will be described in relation to two possible embodiments (FIGS. 5 and 6) of the container that can be used by the apparatus and for the implementation of the method. The two containers are only possible examples of embodiment, not limitative of other solutions, since the apparatus and the method may be implemented with other types of containers.

The apparatus (1) in question can perform treatments for washing, disinfection, sterilization, drying and final packaging of an endoscope inside a container (2). Reduced to its essential structure and with reference to the attached exemplary drawings, a container (2) for performing treatments according to the present invention is provided with connection means able to allow a connection with the apparatus (1). In the non-limiting example illustrated, the container (2) comprises two complementary and separable parts (21, 20) suitable for delimiting an inner compartment in which an endoscope can be positioned.

In the present description, one of the two parts will be called base (20) and the other cover (21). On the base (20) a multi-connector (4) is mounted that allows, as further indicated below, to connect the endoscope channels with a system of external pumps through which washing and sterilization fluids are fed into the same channels. The pumps are supported by the apparatus (1) and are connected to corresponding ducts that can be connected to the multi-connector (4). The multiple connector (4) also allows to feed, by means of corresponding connection means presented by said apparatus (1), a gas for emptying or drying said channels.

Said connector (4) comprises a plurality of external couplings (40) each of which is connected to a respective internal bush (41) by means of a corresponding tube (42) and respective fittings (43). The external couplings (40) are provided on the external side of the part (21), while the bushings (41) are on the inner side of the same part (21). In practice, through the multi-connector (4) the apparatus (1) is able to perform the washing, disinfection, sterilization and drying of the internal parts of the endoscope in a safe, rapid and effective manner.

In FIG. 5 the multiple connector (4) is shown schematically and in different scales of magnification both from the outside (detail on the left) and from the inside (detail on the right), with possible connections. The block (MM) schematically represents the various components, such as, for example, pumps, sensors, circuits, actuators, timers, etc., which are part of the apparatus (1) and are connected by means of the ducts (45) to the external couplings (40). The components (MM) can be activated through a central unit (UC) delegated to the monitoring and the control of the apparatus (1). In FIG. 5 are shown the connections (46) between the central unit (UC) and the components of the apparatus (MM) delegated to the internal treatment of the container (2) and of the endoscope contained therein, and the connections (46′) with possible means (LE) suitable for the external washing of the container (2). The components (MM) of the apparatus (1) are also connected to connectors (48, 49) (better described below) for the treatment of the external parts of the endoscope (EN) and of the inner walls of the container (2).

In the right-hand detail, which shows the inside of the connector (4), the connection (44) of the inner bushings or fittings (41) is shown schematically with the endoscope (EN).

In other words, the apparatus (1), through its components (MM) and the connection defined by the elements (45)-(40)-(41)-(44), can be connected to the internal channels of the endoscope (EN) for the execution of the treatments to be performed. Moreover, through the connection (45′), its components (MM) can be connected to the connectors (48, 49).

The association between the base (20) and the cover (21) can be carried out in various ways, for example by means of hinges and circular clamps. In FIG. 5 a circular clamp (6) is shown, formed by two substantially semi-circumferential parts fixed at a first end to a hinge and provided at the other end, or distal end, with a stable connection means, which in the example is constituted by a hook element (63) and a corresponding lever element (62) allowing the container (2) to be closed.

The base (20) and the cover (21) can be provided with corresponding peripheral edges on which the circular clamp (6) is fitted, preferably with a sealing gasket, the clamp having an internal profile complementarily shaped with respect to said edges. In practice, the closure of the container (2) provides for the approach of the base and the cover, the covering of the edges (23) through the two parts of the clamp (6) and the locking of the latter thanks to the interaction between the ring-shaped part of the lever element with the hook element (63) and the activation of the lever element (62). The connection so realized between the base and one of the covers makes the container (2) impermeable to fluids, including air, making the positioning of the endoscopes inside the containers as well as their subsequent pickup safe, easy and effective. Obviously the materials chosen for the construction of the clamp (6) and its parts will be suitable to allow a stable closure.

The circular clamp (6) is also provided with a handle (64) which, in the illustrated examples, is pivoted to the body of the clamp itself, being hinged about an axis which is substantially tangential with respect to the circular profile of the clamp (6). The handle (64) may also be made differently as shown in the schematic example of FIG. 6.

As anticipated above, in addition to the multi-connector (4), the container (2) is also provided with openings to allow the fluids to entry and exit the container. In the drawing, two openings (49) are provided in the area of the container (2) which in use will be arranged on top, while an opening (48) is disposed in the diametrically opposed zone, that is, the zone that in use will be placed at the bottom. Inside the container (2), for example (but not exclusively) in correspondence of the openings, means can be advantageously provided for diverting the fluids at the inlet to increase the efficiency of the treatment. Through the aforementioned openings it is possible to fill (and empty) the inside of the container (2) to allow cleaning/sterilization/drying of the external parts of the endoscope and the internal parts of the container. The container (2) is advantageously provided with an internal pressure indicator (7) able to display anomalies related to lower pressure values with respect to a predetermined alarm value.

This characteristic is particularly important because it allows to have a substantially error-free indicator of the sterility preservation. In other words, at the end of the washing, sterilization and drying treatments, which may take place in the apparatus (1), it is possible to pressurize the inside of the container (2), i.e. to bring the pressure to a higher value than the atmospheric pressure, and then monitor the internal pressure value in order to provide a visual alarm signal through the indicator (7). The indicator (7) can consist of a pressure gauge or an indicator with an index that moves proportionally to the internal pressure. It can also emit visual and/or acoustic signals.

In view of above, it is understood that the present invention starts from an assumption which is opposite to systems which provide for the maintenance of sterilized instruments at the atmospheric pressure, isolated from the outside only by means of containers provided with filters or special packaging or casing of a known type. In fact, a possible loss in a container or the damage to a package causes the entry of fluids inside it with the possibility of introducing also pathogenic germs and similar particles that are not allowed; on the contrary, the natural loss of pressure in a pressurized container would imply the escape of gas, avoiding the entry of external agents, maintaining the sterility condition inside it until the achievement of an equality between the external and internal pressures.

Another feature of the present invention relates to the possible presence of a support structure (8) of the container (2) capable of allowing the vertical positioning of the container (2) while maintaining the axisymmetric conformation of the container (2), i.e. without varying shape. The support structure (8), which is shown schematically in FIG. 6, is formed by two plates which can be fixed to the two sides of the container (2). The plates (8) are provided with resting feet (80) for vertical positioning on a horizontal plane (T) and can be provided with a perforated portion (not shown) suitable for allowing the passage of the multiple connector (4). In practice, the structure (8) allows the container (2) to be arranged vertically, allowing access both to the multiple connector (4) and to the upper (49) and bottom (48) openings.

The structure (8) and the handle (64) make the handling of the container extremely simple and safe for the operators.

As previously stated, the present invention relates to an apparatus and a method for washing, disinfecting, sterilizing and drying endoscopes and similar instruments; a non-limiting example of the apparatus (1) is shown in FIGS. 1-4.

The apparatus (1) has a structure comprising one or more modules (10) suitable for receiving the container (2). Each module is provided with a pair of doors (11, 11′) hinged on a horizontal axis so as to be positioned on at least one closing configuration, in which the door (11, 11′) is substantially vertical, and on a opening configuration, in which the door (11, 11′) is substantially horizontal, forming an approximately 90° angle with the vertical side of the apparatus (1).

The apparatus (1) can therefore comprise a main part and a series of modules so as to provide a modularity allowing the construction of machines of different capacities according to the operating requirements. In the figures the main module which contains the central unit (UC) and which is arranged on the left in FIG. 1 is marked with (100), the modules which can be added to form the apparatus (1) are marked with (10). In the illustrated example, the apparatus (1) is provided with three modules (10). Obviously the number of modules (10) may be different depending on the design choices of the apparatus (1).

One (11) of the doors (11, 11′) is placed upstream, i.e. in an area, marked with (A) in the drawings, where a certain degree of contamination is expected, while the other door (11′) is placed downstream, i.e. in an area, marked with (B) in the drawings, where a degree of sterility is expected, usually higher than that of zone (A). By way of example, zone (A) may be a preliminary washing area of the instruments, while zone (B) is a sterile area. In other words, a passage called “trough the wall” is realized, that is a passage between two zones with a different degree of sterility.

The present description refers to the non-limiting drawings attached. In other embodiments of the invention, for example, the apparatus may be provided with a single door that can be used both for loading and unloading the container (2). In this case there will not be the differentiated zones (A) and (B) just described.

Each door (11, 11′) is provided with guides (13) suitable for housing the container (2), preferably but not exclusively equipped with the support structure (8). In addition or alternatively to the guides (13), a guide structure (12) can also be provided, also designed to house the container (2). The system can also provide an automatic door opening, even with vertical sliding.

During treatment, a container (2) with an endoscope inside is placed on the upstream door (11) that has been previously brought into the opening configuration. The downstream door (11′) is closed. The container (2) is then introduced into the apparatus (1) where appropriate connectors connect the operative components of the apparatus such as pumps, sensors, etc. to the corresponding connection means (4, 48, 49) of the container (2). The upstream door (11) is closed.

The apparatus (1) provides, using the multiple connector (4), to perform in succession the internal washing treatment of the endoscope, its disinfection/sterilization and subsequent drying. Similarly, through the accesses (48, 49), it performs the external treatment of the endoscope and of the inside of the container (2). Furthermore, it can provide for external washing of the container (2) by means of nozzles or similar means for dispensing a disinfectant to increase also the degree of external sterility of the same container (2). The means for washing the container are not illustrated in detail but they are schematically represented by the block (LE) of FIG. 5.

As previously described, for carrying out the washing, sterilization and drying phases, the apparatus (1) is provided with suitable means (schematically represented by the block MM) such as pumps, vacuum pumps, connectors, fittings, valves or other means of regulation also automatic of flows, control and command circuits, sterilizing fluids tanks and vectors, and will be connected to the corresponding power and supply networks.

Furthermore, the apparatus (1) may be provided with automatic or semi-automatic means for connection to the container (2) and for its movement from upstream to downstream, i.e. from the area (A) to the area (B). In practice, the connection of the apparatus (1) to the container (2) can be carried out by an operator or by the apparatus in an automated manner, or it may include manual and automated steps. Similarly the movement can be manual, automatic or semi-automatic.

The container (2) can be made of a material suitable to resist to both positive and negative pressures and chemical agents.

Advantageously, the apparatus (1) can be used for the sterilization of endoscopes and similar instruments, and is of the type comprising connecting means for a container inside which the endoscope to be sterilized is placed, said means being suitable for introducing into said container one or more washing and/or sterilizing fluids and one or more fluids for drying the endoscope. In practice it is an apparatus that not only sterilizes, but also performs the washing and drying of the endoscopes inside the container (2). This apparatus does not require sterile packaging operations that instead characterize the state-of-the-art systems that are not able to perform the complete treatment of the instrument.

The apparatus (1) is provided with means for pressurizing said container (2), that is to pressurize it at a pressure higher than atmospheric pressure. The means for carrying out the pressurization can be pumps or other means suitable for this purpose.

The container (2), before being released by the apparatus (1), is brought to a pressure higher than atmospheric pressure.

In particular, the pressure inside the container (2) is preferably brought to a value of 250-350 mbar (250-350 hPa, i.e. 25000-35000 Pa) higher than atmospheric pressure i.e. higher than the atmospheric pressure of the environment in which the pressurized container (2) is stored.

With the apparatus (1) it is therefore possible to carry out a method for the sterilization of endoscopes and similar instruments, of the type comprising the washing and/or disinfection/sterilization of an endoscope inside a container by one or more washing fluids, and/or the subsequent drying by one or more drying fluids. The method is characterized by the fact that the container (1) is pressurized at the end of the drying phase, i.e. before its release, that is, determining within it a pressure higher than the atmospheric pressure.

The apparatus (1), again by means of the aforementioned connections (4, 48, 49) and its components suitable for the purpose, is further provided with means capable of determining a pressure inside said container (1) considerably lower than atmospheric; pressure that is apt, in addition, to increasing the drying efficiency and reducing drying times. The means for carrying out the depressurization can be pumps or other means suitable for the purpose.

A method and an apparatus usable with a container (2) according to the invention can determine within the container a pressure considerably lower than atmospheric pressure, for example lower than 20 mbar or lower than 10 mbar, or even at lmbar. In other words, the pressure (in absolute value) inside the container is less than 2000 Pa, preferably, 1000 Pa, or even 100 Pa.

In this way the drying time is drastically reduced.

Depressurization is also advantageous in the sterilization phase because, thanks to the depressurization, the sterilizing agent diffuses into the container (2) more effectively.

In practice, the drying time is decreased because the significantly lower pressure causes a decrease of the time necessary for evaporation. Likewise, the significantly lower pressure allows to convey the chemical agents used for sterilization in nebulized or vaporized form.

Another advantageous feature of the invention lies in the possibility of subdividing the sterilizer dispensing step over time. In fact, the sterilizer (which is normally in liquid phase) is dispensed in a vaporized state in successive phases, subdividing the total delivered quantity in several successive steps. The so-called “pulsed vacuum” is thus created, that is a series of temporal intervals in which the vacuum is produced (more precisely a strong depressurization) which determine, with the same consumption of the sterilizing fluid, a more effective sterilizing action and a total consumption substantially lower if compared to a machine that does not use vacuum. Also the phase of the depressurization for drying is improved by using the pulsed vacuum for this phase. In fact, when this phase is divided into intervals, the effectiveness of the flow rate in the drying itself is increased.

The pulsed vacuum is also effective because it determines a sort of “washing” of the ducts with the removal of any material present in them.

In particular, pressurization and depressurization, even pulsed, are driven and controlled by said command and control unit (UC).

Therefore, according to the invention as described and illustrated, a method for the sterilization of endoscopes and similar instruments comprises the following steps of: washing and/or disinfecting or sterilizing an endoscope inside a container by one or more washing fluids; drying the endoscope inside the container using one or more drying fluids. The pressure inside the container during disinfection or sterilization and/or drying is kept lower than atmospheric pressure and the container is pressurized after drying, at a pressure higher than atmospheric pressure, before releasing the endoscope.

Moreover, the method provides the pressurization of the container by increasing the internal pressure of the container with respect to atmospheric pressure of a value between 250 and 350 mbar, corresponding to 25,000-35,000 Pa.

The method can provide for external washing of the container (2).

An apparatus for the implementation of the method described above is of the type comprising means for washing, disinfecting, sterilizing and drying endoscopes positioned inside a container, as well as connecting means for connecting the container inside the apparatus, the instrument to be sterilized is placed, suitable for feeding into said container one or more washing and/or sterilizing fluids and one or more fluids for drying the endoscope. The apparatus is provided with pressurization means able to pressurize said container, i.e. to determine inside it a pressure higher than atmospheric pressure, the apparatus (1) also comprising command and control means (UC) which control and command said pressurization means adapted to activate said pressurization means after the activation of the drying means.

The apparatus may comprise washing means (LE) suitable for carrying out a washing of the exterior of the container as well as means for decreasing the pressure inside said container, or adapted to determine inside it a pressure lower than atmospheric pressure.

The driving and control means (UC) of the apparatus can activate said means for decreasing the pressure inside said container during the sterilization and/or drying phase. Furthermore, the apparatus may have a modular structure composed of a central unit (100) and one or more modules (10) provided with means for housing a container (2) and connectable to said central unit (100).

The values indicated above are not limiting for other embodiments of the invention. Moreover, the execution details may in any case vary in the form, dimensions, arrangement of the elements, nature of the materials used, without however departing from the scope of the idea of solution adopted or of the inventive concept and therefore remaining within the limits of the protection granted by the present patent.

1. A method for sterilization of endoscopes and similar instruments, comprising the successive steps of:
washing and/or disinfecting or sterilizing an endoscope inside a container by means of one or more washing fluids;
dry the endoscope within the container by means of one or more drying fluids, method characterized in that the pressure inside said container during disinfection or sterilization and/or drying is kept below the atmospheric pressure and the container is pressurized after the drying, at a pressure higher than the atmospheric pressure, before releasing the endoscope.

2. The method according to claim 1, characterized by pressurizing the container by increasing the internal pressure of the container with respect to atmospheric pressure at a value between 250 and 350 mbar, corresponding to 25000 to 35000 Pa.

3. The method according to claim 1, wherein an external washing of the container is carried out.

4. An apparatus for implementing the method of claim 1, apparatus of the type comprising means for washing, disinfecting, sterilizing and drying endoscopes disposed within a container, as well as connection means for connecting to the apparatus the container within which the instrument to be sterilized is placed and capable of introducing into said container one or more washing and/or sterilizing fluids and one or more endoscope drying fluids, apparatus characterized in that it is provided with pressurizing means for pressurizing said container, that is to determine within it a pressure higher than atmospheric pressure, said apparatus further comprising control and command means which control and drive said pressurization means for activating said pressurization means subsequently to the activation of the drying means.

5. The apparatus according to claim 4, wherein said means able to pressurize the container increase the internal pressure of the container with respect to atmospheric pressure of a value between 250 and 350 mbar, corresponding to 25000-35000 Pa.

6. The apparatus according to claim 4, wherein it comprises washing means (LE) suitable for carrying out a washing of the exterior of the container.

7. The apparatus according to claim 4, wherein it is further provided with means for decreasing the pressure inside said container, i.e. adapted to determine inside it a pressure lower than atmospheric pressure.

8. The apparatus according to claim 5, wherein said command and control means activate said means for decreasing the pressure inside said container during the sterilization and/or drying phase.

9. The apparatus according to claim 4, wherein it has a modular structure composed of a central unit and of one or more modules provided with means for receiving a container and connectable to said central unit.
